(19) United States
(12) Patent Application Publication
(10) Pub. No.: US 2026/0363892 A1
(43) Pub. Date: Nov. 19, 2026

(54) ABDOMINAL BINDER AND BACK SUPPORT MEMBER

(71) Applicant: Shenzhen Lute Jiacheng Supply Chain Management Co., Ltd., Shenzhen (CN)

(72) Inventors: Lan Li, Shenzhen (CN); Zijuan Qi, Shenzhen (CN); Ping Yan, Shenzhen (CN)

(21) Appl. No.: 19/220,135

(22) Filed: May 28, 2025

(30) Foreign Application Priority Data
May 16, 2025 (CN) 202520976444.2

(51) Int. Cl. A61F 5/03

(52) U.S. Cl. CPC A61F 5/03

(57) ABSTRACT

The present application discloses an abdominal binder, comprising: a waist support portion; a first abdominal compression portion connected to a first side of the waist support portion; a second abdominal compression portion connected to a second side of the waist support portion; the first abdominal compression portion and the second abdominal compression portion being detachably connected to each other; and a back support member disposed on the waist support portion, the back support member comprising at least one support strip, the support strip being configured to extend in a height direction of the user, and being gradually curved from both ends toward the center in a direction approaching the user's back.

### CROSS-REFERENCE TO RELATED APPLICATIONS

The present application claims priority to Chinese Patent Application No. 202520976444.2, filed on May 16, 2025, the entire contents of which are incorporated herein by reference.

### TECHNICAL FIELD

The present application relates to the technical field of maternal and baby products, particularly to an abdominal binder and back support member.

### BACKGROUND

The abdominal binder is a commonly used wearable assistive device that is widely applied in scenarios such as body shaping, figure enhancement, and postoperative rehabilitation. Conventional abdominal binders typically provide abdominal support and shaping effects through elastic materials and compression-based designs. However, such traditional designs exhibit certain deficiencies during actual use, particularly in terms of supporting force and wearing comfort.

Most abdominal binders currently available on the market provide relatively weak support for the waist and back, relying solely on elastic materials or simple reinforcement structures, which makes it difficult to offer sufficient support to the user. Under such circumstances, especially during prolonged wear, the user may experience fatigue in the waist and back, adversely affecting daily activities and overall comfort. In addition, the design of the back support structure is often relatively simplistic and fails to conform to the natural curvature of the human back, resulting in a stiff or uncomfortable wearing experience that further reduces the practicality of the product.

Accordingly, how to design an abdominal binder capable of providing enhanced supporting force while improving back comfort has become a technical problem that urgently needs to be addressed by those skilled in the art.

### SUMMARY

In view of deficiencies in the prior art, an embodiment of the present application provides an abdominal binder and a back support member capable of solving problems of poor back support strength and poor comfort in abdominal binders.

To solve the above technical problems, one embodiment of the present application provides an abdominal binder, comprising:
a waist support portion configured to be positioned in a waist region of a user and to extend laterally to both sides of the user when worn;
a first abdominal compression portion connected to a first side of the waist support portion and configured to extend along an abdominal region of the user when worn;
a second abdominal compression portion connected to a second side of the waist support portion and configured to extend along the abdominal region of the user when worn, wherein the first abdominal compression portion and the second abdominal compression portion are detachably connected to each other and, when connected, provide a compressive force to the abdominal region of the user;
a back support member disposed on the waist support portion, the back support member including at least one support strip extending in a height direction of the user when the abdominal binder is worn, the support strip being gradually curved from both ends toward the center in a direction approaching the user's back.

In one embodiment, the back support member comprises at least a first support strip and a second support strip, the first and second support strips being respectively located on opposite sides of the user's spine when the abdominal binder is worn, and each of the first and second support strips being gradually curved from both ends toward the center in a direction approaching the user's spine.

In one embodiment, each support strip gradually decreases in thickness from the center toward both ends.

In one embodiment, the back support member further comprises connecting strips connected between adjacent support strips.

In one embodiment, the connecting strips comprise at least a first connecting strip and a second connecting strip, the first and second connecting strips being disposed at different positions along the height direction when the abdominal binder is worn.

In one embodiment, the back support member is embedded in the waist support portion.

In one embodiment, the waist support portion is at least partially made of a sheet material, the sheet material comprising a first outer layer, a second outer layer, and a bonding layer disposed between the first and second outer layers.

In one embodiment, the bonding layer comprises a first adhesive sub-layer, a cushioning layer, and a second adhesive sub-layer sequentially laminated from the inner side to the outer side.

In one embodiment, the back support member is embedded in the cushioning layer.

In one embodiment, the cushioning layer is a sponge layer.

In one embodiment, the second outer layer is provided with ventilation holes.

In one embodiment, the first abdominal compression portion comprises at least a first compression band and a second compression band, the first and second compression bands being disposed side by side and extending transversely along the abdominal region when worn, the first and second compression bands being configured to be independently detachably connected to the second abdominal compression portion, and each of the first and second compression bands being independently adjustable in tightness by changing its connection position with the second abdominal compression portion.

In one embodiment, the back support member is disposed on an outer surface of the waist support portion.

In one embodiment, an upper edge of each support strip is arc-shaped.

In one embodiment, a lower edge of each support strip is arc-shaped.

To solve the above technical problems, one embodiment of the present application provides a back support member for an abdominal binder, comprising at least one support strip configured to extend in a height direction of a user when the abdominal binder is worn, the support strip being gradually curved from both ends toward the center in a direction approaching the user's back.

In one embodiment, the back support member comprises at least a first support strip and a second support strip, the first and second support strips being respectively located on opposite sides of the user's spine when the abdominal binder is worn, and each of the first and second support strips being gradually curved from both ends toward the center in a direction approaching the user's spine.

In one embodiment, each support strip gradually decreases in thickness from the center toward both ends.

In one embodiment, the back support member further comprises connecting strips connected between adjacent support strips.

In one embodiment, the connecting strips comprise at least a first connecting strip and a second connecting strip, the first and second connecting strips being disposed at different positions along the height direction when the abdominal binder is worn.

The embodiment of the present application sets the abdominal binder to include: the waist support portion configured to be positioned in the waist region of the user and to extend laterally to both sides of the user when worn; the first abdominal compression portion connected to a first side of the waist support portion and configured to extend along the abdominal region of the user when worn; the second abdominal compression portion connected to a second side of the waist support portion and configured to extend along the abdominal region of the user when worn; the first abdominal compression portion and the second abdominal compression portion being detachably connected and, when connected, providing a compressive force to the abdominal region of the user; and the back support member disposed on the waist support portion, including at least one support strip extending in a height direction of the user when the abdominal binder is worn, the support strip being gradually curved from both ends toward the center in a direction approaching the user's back. The middle portion of the support strip provides stronger support force to increase comfort. Due to the support force applied to the back by the support strip, a reactive force causes the first abdominal compression portion and the second abdominal compression portion to provide greater compressive force to the abdominal region of the user when connected, thereby enhancing the abdominal shaping effect.

### BRIEF DESCRIPTION OF THE DRAWINGS

To more clearly illustrate the technical solutions of the embodiments of the present application or the prior art, a brief introduction of the drawings used in the description of the embodiments or the prior art is provided below. It is apparent that the drawings described below only illustrate some embodiments of the present application. For those skilled in the art, other drawings may be obtained based on the structures shown in these drawings without any creative effort.

FIG. 1 is a schematic structural diagram of an abdominal binder according to an embodiment of the present application viewed from a first perspective in a worn state;

FIG. 2 is a schematic structural diagram of a back support member according to an embodiment of the present application viewed from a first perspective;

FIG. 3 is a schematic structural diagram of the back support member according to an embodiment of the present application viewed from a second perspective;

FIG. 4 is a schematic structural diagram of an abdominal binder according to an embodiment of the present application viewed from the outer surface in an unfolded state;

FIG. 5 is a schematic structural diagram of the abdominal binder according to an embodiment of the present application viewed from the inner surface in an unfolded state;

FIG. 6 is a schematic structural diagram of the abdominal binder according to an embodiment of the present application viewed from a second perspective in a worn state;

FIG. 7 is a schematic structural diagram of an abdominal binder according to another embodiment of the present application viewed from the outer surface in an unfolded state;

FIG. 8 is a schematic diagram illustrating the laminated structure of a sheet material according to an embodiment of the present application;

FIG. 9 is a schematic diagram illustrating the laminated structure of a sheet material according to another embodiment of the present application.

The achievement of the objectives, functional features, and advantages of the present application will be described in detail in combination with the embodiments and with reference to the accompanying drawings.

### DETAILED DESCRIPTION OF THE EMBODIMENTS

In the present application, the terms “arranged,” “provided with,” and “connected” should be understood broadly. For example, a connection may be fixed, detachable, or integrally constructed; it may be mechanical or electrical; it may be direct or indirectly connected through an intermediary, or it may refer to internal communication between two devices, components, or constituent parts. Those of ordinary skill in the art can understand the specific meanings of these terms in the context of the present application based on specific circumstances.

The terms “center,” “longitudinal,” “transverse,” “length,” “width,” “thickness,” “upper,” “lower,” “front,” “rear,” “left,” “right,” “vertical,” “horizontal,” “top,” “bottom,” “inner,” “outer,” “radial,” and “circumferential” are used to describe orientations or positional relationships based on the orientations or positional relationships shown in the accompanying drawings. These terms are used solely for the purpose of facilitating the description of the present application and simplifying the description, and do not indicate or imply that the referenced devices or components must have a specific orientation or be constructed and operated in a specific orientation. Therefore, these terms should not be construed as limiting the present application.

Furthermore, the terms “first” and “second” are used for descriptive purposes only and should not be construed as indicating or implying relative importance or implicitly specifying the quantity of the indicated technical features. Thus, features defined by “first” or “second” may explicitly or implicitly include at least one such feature. In the description of the present application, the term “plurality” means at least two, such as two, three, etc., unless explicitly stated otherwise.

Additionally, some of the above terms may be used to indicate meanings other than orientation or positional relationships. For example, the term “upper” may, in some cases, also be used to indicate a certain dependency or connection relationship. Those of ordinary skill in the art can understand the specific meanings of these terms in the context of the present application based on specific circumstances.

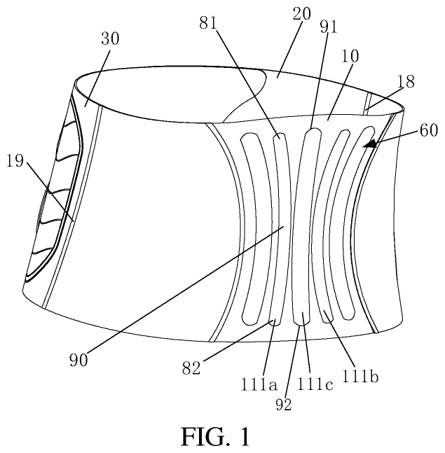

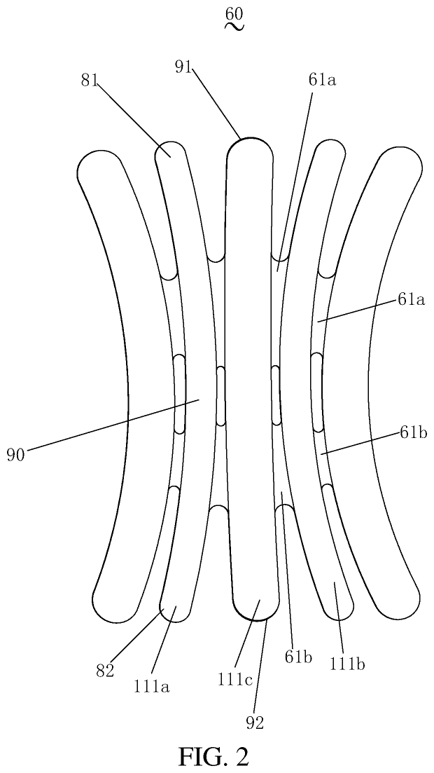

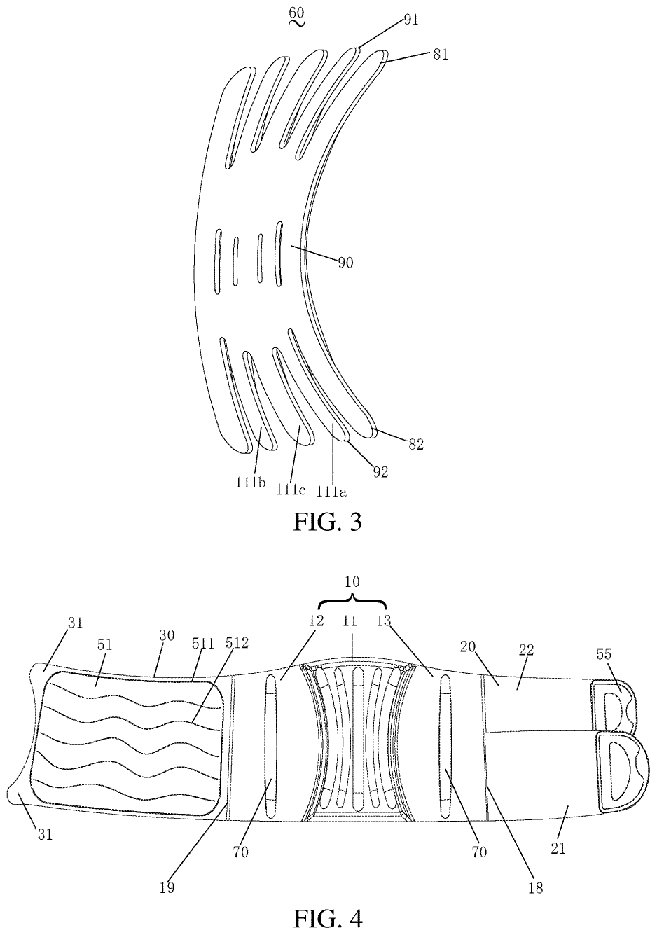

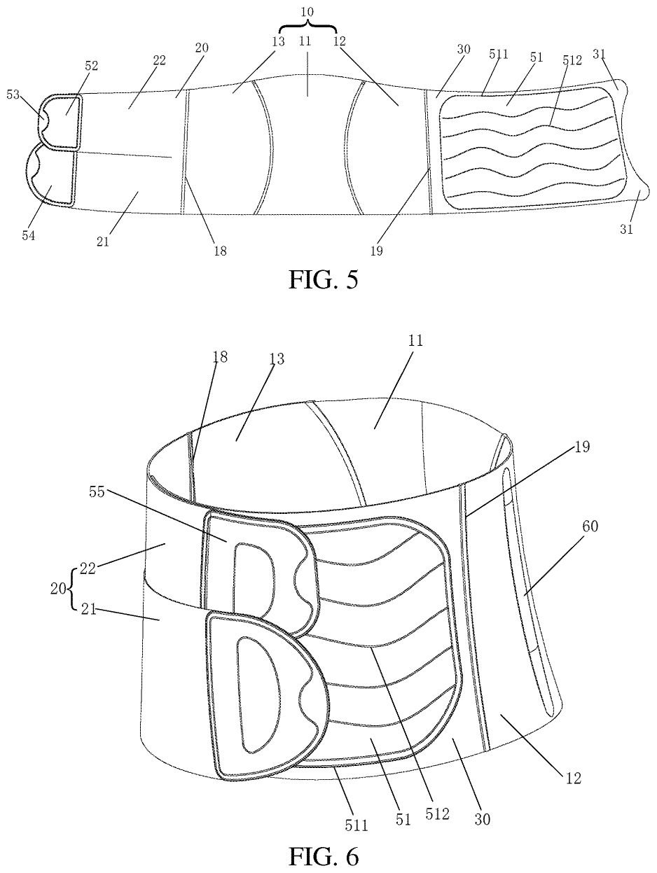

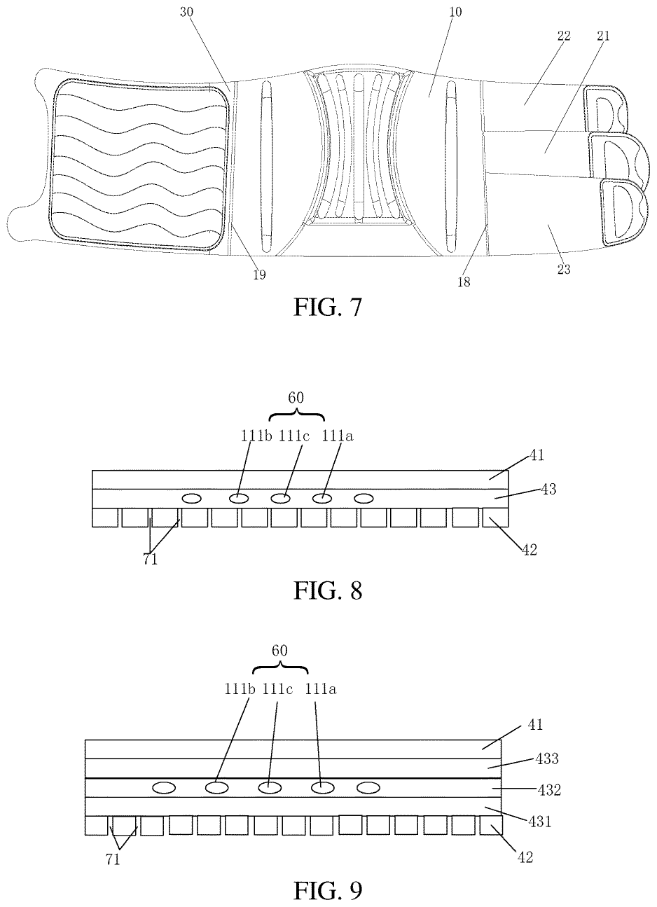

Please refer to FIGS. 1 to 9. FIG. 1 is a schematic structural diagram of an abdominal binder according to an embodiment of the present application viewed from a first perspective in a worn state; FIG. 2 is a schematic structural diagram of a back support member according to an embodiment of the present application viewed from a first perspective; FIG. 3 is a schematic structural diagram of the back support member according to an embodiment of the present application viewed from a second perspective; FIG. 4 is a schematic structural diagram of an abdominal binder according to an embodiment of the present application viewed from the outer surface in an unfolded state; FIG. 5 is a schematic structural diagram of the abdominal binder according to an embodiment of the present application viewed from the inner surface in an unfolded state; FIG. 6 is a schematic structural diagram of the abdominal binder according to an embodiment of the present application viewed from a second perspective in a worn state; FIG. 7 is a schematic structural diagram of an abdominal binder according to another embodiment of the present application viewed from the outer surface in an unfolded state; FIG. 8 is a schematic diagram illustrating the laminated structure of a sheet material according to an embodiment of the present application; and FIG. 9 is a schematic diagram illustrating the laminated structure of a sheet material according to another embodiment of the present application.

As shown in FIG. 1, FIG. 4, FIG. 5, and FIG. 6, in one embodiment of the present disclosure, the abdominal binder comprises a waist support portion 10, a first abdominal compression portion 20, and a second abdominal compression portion 30. The waist support portion 10 is configured to be positioned in the waist region of the user and to extend laterally to both sides of the user when worn. The first abdominal compression portion 20 is connected to a first side 18 of the waist support portion 10 and configured to extend along the abdominal region of the user when worn. The second abdominal compression portion 30 is connected to a second side 19 of the waist support portion 10 and configured to extend along the abdominal region of the user when worn. The first abdominal compression portion 20 and the second abdominal compression portion 30 are detachably connected to each other, and when connected, provide a compressive force to the abdominal region of the user. The tightness of the first abdominal compression portion 20 and the second abdominal compression portion 30 can be adjusted by changing the detachable connection position. The detachable connection means include, but are not limited to, hook-and-loop fasteners, hook-and-eye connection structures, snap fasteners, and the like.

As shown in FIG. 1, FIG. 2, and FIG. 3, the back support member 60 is disposed on the waist support portion 10. The back support member 60 may be embedded within the waist support portion 10 or disposed on the outer surface of the waist support portion 10; the embodiments of the present disclosure do not limit this arrangement. The hardness of the back support member 60 is greater than that of the waist support portion 10, the first abdominal compression portion 20, and the second abdominal compression portion 30. The back support member 60 is made of a rigid material and has elasticity. Optionally, the back support member 60 is made of TPU (Thermoplastic Polyurethane), hard silicone, or carbon fiber composite material.

As shown in FIG. 1, FIG. 2, and FIG. 3, the back support member 60 comprises at least one support strip 111a, 111b, 111c, which extend in the height direction of the user when the abdominal binder is worn. The support strips 111a, 111b, and 111c are gradually curved from both ends 81,82 toward the center 90 in a direction approaching the user's back when the abdominal binder is worn. The support strips 111a, 111b, and 111c, by gradually bending from the ends to the center toward the user's back, can closely fit the natural curvature of the user's spine, especially the muscle groups on both sides of the spine. This design avoids the uneven distribution of support force caused by traditional flat back support members, ensuring that the support force precisely acts on key areas of the back, effectively relieving spinal pressure. The number of support strips shown in the figures is multiple; however, it should be understood that the number of support strips may be one or two, and the embodiments of the present disclosure do not limit this.

As shown in FIG. 1, FIG. 2, and FIG. 3, in one embodiment, the back support member 60 at least comprises a first support strip 111a and a second support strip 111b, wherein the first and second support strips 111a, 111b are respectively located on opposite sides of the user's spine when the abdominal binder is worn, and each of the first and second support strips 111a, 111b is gradually curved from both ends 81,82 toward the center 90 in a direction approaching the user's spine. The support strip 111c extends substantially along the direction of the user's spine when the abdominal binder is worn. The first and second support strips 111a, 111b precisely fit the natural curvature of the spine, providing symmetrical and even support. This design effectively disperses pressure on the spine, reducing the burden on the spine and surrounding muscles, and is particularly suitable for users who need to stand or be active for long periods.

As shown in FIGS. 1, 2, and 3, in one embodiment, the support strips 111a, 111b, and 111c gradually decrease in thickness from the center 90 toward both ends 81, 82. The thicker middle portion of the support strips provides the primary support force, ensuring effective support for the spine and the surrounding muscle groups, while the gradually thinning design at both ends 81, 82 achieves a smooth transition of the support force. This design avoids local pressure concentration caused by abrupt thickness changes, enabling even distribution of the support force and reducing pressure on the skin, thereby significantly enhancing wearing comfort. The thinning thickness at both ends 81, 82 of the support strips makes the edges of the abdominal binder softer and lighter, reducing the sense of pressure and friction when in contact with the skin. This design is especially suitable for scenarios involving prolonged wear, effectively preventing skin irritation caused by the edges of conventional support members and significantly improving skin comfort. Moreover, the thinning at both ends 81, 82 reduces the overall weight of the abdominal binder, making it lighter and more suitable for extended wear. This design is particularly beneficial for pregnant women, postpartum women, and individuals requiring lumbar and back support, significantly enhancing the wearing experience.

As shown in FIG. 2, in one embodiment, the back support member 60 further comprises connecting strips 61a and 61b, which are connected between adjacent support strips 111a and 111b. In one embodiment, connecting strips are provided between every two adjacent support strips to connect the entire back support member 60 into an integral structure. The connecting strips serve both to enhance the overall support strength and stability and to facilitate the integral injection molding of the back support member 60.

As shown in FIG. 2, in one embodiment, the connecting strips 61a and 61b include at least a first connecting strip 61a and a second connecting strip 61b, wherein the first connecting strip 61a and the second connecting strip 61b are arranged at different positions along the height direction when the abdominal binder is worn.

In one embodiment, the back support member 60 is embedded within the waist support portion 10. By embedding the back support member 60 in the waist support portion 10, discomfort caused by direct contact between the back support member 60 and the user's body can be avoided, with the waist support portion 10 providing a certain cushioning effect.

As shown in FIG. 8, in one embodiment, the waist support portion 10 is at least partially made of a sheet material 40, the sheet material 40 comprising a first outer layer 41, a second outer layer 42, and a bonding layer 43 disposed between the first outer layer 41 and the second outer layer 42.

As shown in FIG. 9, in one embodiment, the bonding layer 43 comprises a first adhesive sub-layer 431, a cushioning layer 432, and a second adhesive sub-layer 433 sequentially laminated from the inner side to the outer side.

In one embodiment, the back support member (not shown) is embedded in the cushioning layer 432. Embedding the back support member in the cushioning layer enhances the cushioning effect of the waist support portion 10 on the back support member.

In one embodiment, the cushioning layer 432 is a sponge layer. The sponge layer has excellent elasticity and softness, providing comfortable cushioning when worn by the user and reducing pressure on the waist and back. Meanwhile, the lightweight characteristic of the sponge layer does not increase the overall weight of the abdominal binder, making it suitable for prolonged use. Additionally, the sponge layer has superior breathability, effectively wicking away sweat to keep the skin dry, further enhancing wearing comfort.

In one embodiment, the second outer layer 42 is provided with ventilation holes 71. The design of the ventilation holes 71 significantly improves the breathability of the abdominal binder, promoting air circulation and preventing stuffiness and discomfort caused by prolonged wear. The distribution of the ventilation holes 71 can be optimized according to the user's needs to ensure optimal ventilation in key areas such as the waist and back. This design is especially suitable for use in hot environments or during high-intensity activities, greatly enhancing the user experience.

As shown in FIGS. 1, 2, and 3, in one embodiment, the upper edges 91 of the support strips 111a, 111b, and 111c are arc-shaped. The arc-shaped design reduces contact pressure between the support strips and the user's back, avoiding friction or pressure from the edges on the skin. This design is particularly suitable for prolonged wear and can significantly enhance skin-contact comfort. In addition, the curved upper edges 91 contribute to the aesthetic appeal of the support strips, giving the abdominal binder a smoother and more natural overall appearance.

As also shown in FIGS. 1, 2, and 3, in one embodiment, the lower edges 92 of the support strips 111a, 111b, and 111c are arc-shaped. The arc-shaped design of the lower edges 92 complements the upper edges 91 and further improves the fit between the support strips and the user's body. This design prevents pressure from the lower ends of the support strips on the waist, reducing any foreign body sensation during wear. Meanwhile, the arc-shaped lower edges 92 enhance the dynamic adaptability of the support strips, allowing them to move more flexibly when the user bends or twists, thereby improving the overall wearing experience.

As shown in FIGS. 5 and 6, in one embodiment, the abdominal binder is at least partially made of a sheet material, which comprises a first outer layer 41, a second outer layer 42, and an adhesive layer 43 disposed between the first outer layer 41 and the second outer layer 42. The first outer layer 41 and the second outer layer 42 are bonded together through the adhesive layer 43. Through the above structure, the adhesive layer between the first outer layer 41 and the second outer layer 42 can increase the overall elasticity of the abdominal binder, thereby enhancing the abdominal compression effect, increasing the tightening force, and making the abdominal binder flatter. It can also be freely cut into a desired shape, which facilitates the design of the edge lines of the abdominal binder.

In one embodiment, the adhesive layer 43 comprises, in sequence from inside to outside, a first adhesive sub-layer 431, a cushioning layer 432, and a second adhesive sub-layer 433. In this embodiment, the cushioning layer 432 is embedded within the adhesive layer, which can increase the overall thickness and softness of the abdominal binder, making it softer and more comfortable to wear. In another embodiment, the adhesive layer may consist solely of an adhesive material without the embedded cushioning layer. This embodiment is not limited thereto.

The adhesive material may include hot melt adhesive, polyurethane adhesive, solvent-based glue, silicone adhesive, acrylic adhesive, two-component adhesive, etc., without limitation.

In one embodiment, the cushioning layer 432 is a sponge layer or a gauze layer. The cushioning layer 432 functions to increase the thickness and softness of the abdominal binder, improving wearing comfort.

In one embodiment, the cushioning layer 432 is provided with ventilation holes. For example, the cushioning layer 432 may be a mesh layer. While increasing the thickness, the cushioning layer 432 ensures breathability through the ventilation holes.

In one embodiment, the first outer layer 41 is made of polyester-spandex fabric, and serves as the outermost layer when the abdominal binder is worn, while the second outer layer 42 is made of nylon-spandex fabric and serves as the innermost layer when worn.

In one embodiment, the first adhesive sub-layer 431 and the second adhesive sub-layer 433 are glue layers. The polyester-spandex fabric is a blended or interwoven fabric composed of polyester and spandex, offering properties such as water resistance, abrasion resistance, high elasticity, and wrinkle resistance. The nylon-spandex fabric is an elastic fabric blended or interwoven from nylon and spandex, which combines the toughness of nylon and the high elasticity of spandex. It also provides moisture-wicking properties and is not easily corroded by sweat.

As shown in FIGS. 1, 2, and 3, in one embodiment, the first abdominal compression portion 20 and the second abdominal compression portion 30 are detachably connected by hook-and-loop fasteners. One of the first abdominal compression portion 20 and the second abdominal compression portion 30 is provided with the hook surface of the hook-and-loop fastener, and the other is provided with the loop surface. Optionally, the first abdominal compression portion 20 is connected to the right side of the waist support portion 10, and the second abdominal compression portion 30 is connected to the left side of the waist support portion 10. The loop surface 51 of the hook-and-loop fastener is provided on the second abdominal compression portion 30. The hook surface 52 of the hook-and-loop fastener is provided on the first abdominal compression portion 20. Through the hook-and-loop fastener design, the first abdominal compression portion 20 and the second abdominal compression portion 30 can be easily connected and detached, allowing the user to freely adjust the abdominal compression force as needed. This design increases the flexibility and comfort of the abdominal binder. The hook-and-loop fasteners may be arranged at or near the end portions of the first abdominal compression portion 20 and the second abdominal compression portion 30.

In one embodiment, a notch 53 is formed on the hook-and-loop fastener 52. Optionally, the notch 53 is formed on the hook surface 52 of the hook-and-loop fastener on the first abdominal compression portion 20. By providing the notch 53 on the hook-and-loop fastener 52, friction between the user's fingers and the fastener during wearing is avoided, thereby improving the comfort of the fastening operation.

In one embodiment, the hook-and-loop fastener is stitched to the first abdominal compression portion 20 and/or the second abdominal compression portion 30 by sewing threads. The sewing threads include a first sewing thread 511 located in the edge area of the hook-and-loop fastener 51, and a second sewing thread 512 located in the central area of the hook-and-loop fastener 51. By sewing the hook-and-loop fastener to the first and/or second abdominal compression portion, detachment due to glue failure caused by repeated washing can be avoided. The second sewing thread 512 in the central area can prevent the central portion of the fastener from peeling off due to prolonged pulling.

As shown in FIGS. 1, 2, and 3, in one embodiment, the first abdominal compression portion 20 comprises at least a first compression band 21 and a second compression band 22. The first compression band 21 and the second compression band 22 being disposed side by side and extending transversely along the abdominal region when worn. The first compression band 21 and the second compression band 22 are configured to be detachably connected to the second abdominal compression portion 30 independently and can be independently adjusted in tightness by changing their connection positions with the second abdominal compression portion 30. In this manner, the compression force at the positions of the first compression band 21 and the second compression band 22 can be adjusted in zones, thereby improving the overall wearing comfort of the abdominal binder.

In one embodiment, the first compression band 21 and the second compression band 22 are at least partially overlapped. In this way, the first compression band 21 and the second compression band 22 partially overlap at adjacent positions, which can prevent the user's excess flesh from being squeezed out between the two bands, thereby avoiding pinching of the abdomen and enhancing both comfort and appearance during wear.

As shown in FIGS. 1, 2, and 3, in one embodiment, one of the inner surface of the first compression band 21 and the outer surface of the second compression band 22 is provided with a loop surface of a hook-and-loop fastener, and the other is provided with a hook surface, such that the overlapping areas of the first and second compression bands can be adhered together during wear. Optionally, the inner surface of the first compression band 21 is provided with the hook surface 54, and the outer surface of the second compression band 22 is provided with the loop surface 55. In this way, the first compression band 21 and the second compression band 22 can adhere to each other, thereby enhancing the flatness of the abdominal binder surface when worn.

In one embodiment, the first compression band 21 is positioned at least partially underneath the second compression band 22 during wear, and the length of the first compression band 21 is greater than that of the second compression band 22. By making the lower first compression band 21 longer than the upper second compression band 22, the wearing comfort can be improved.

As shown in FIG. 4, in another embodiment, the first abdominal compression portion 20 further comprises a third compression band 23. The first compression band 21, the second compression band 22, and the third compression band 23 extend laterally along the abdomen side by side when worn. These three compression bands are configured to be independently and detachably connected to the second abdominal compression portion 30 and can be independently adjusted in tightness by changing their respective connection positions. In this manner, the inclusion of three compression bands accommodates users with longer torsos. It should be understood that in other embodiments, four or more compression bands may also be provided, which is not limited herein. Optionally, the length of the third compression band 23 is less than that of the first compression band 21 and greater than that of the second compression band 22, thereby improving wearing comfort and complying with ergonomic design.

As shown in FIGS. 1, 2, and 3, in one embodiment, the abdominal binder is embedded with an anti-roll support strip 70, which extends substantially in the vertical direction when the abdominal binder is worn. In one embodiment, the thickness of the anti-roll support strip 70 gradually decreases from the center 90 toward both ends 81, 82. In another embodiment, the width of the anti-roll support strip 70 gradually decreases from the center 90 toward both ends 81, 82. In this embodiment, the design of the anti-roll support strip 70, with thickness and width gradually tapering from the center 90 to both ends 81, 82, can effectively improve the comfort and fit of the abdominal binder, preventing the ends from being too rigid and affecting comfort. The anti-roll support strip 70 may be made of materials such as thermoplastic polyurethane or silicone.

As shown in FIGS. 1, 2, and 3, in one embodiment, the waist support portion 10 comprises a back panel 11 and a first side panel 12 and a second side panel 13 extending respectively along two sides of the back panel 11. The thickness of the back panel 11 is greater than the thickness of the first side panel 12, and the thickness of the back panel 11 is greater than the thickness of the second side panel 13.

In one embodiment, the thickness of the first abdominal compression portion 20 is greater than the thickness of the first side panel 12, and the thickness of the first abdominal compression portion 20 is greater than the thickness of the second side panel 13. The thickness of the second abdominal compression portion 30 is greater than the thickness of the first side panel 12, and the thickness of the second abdominal compression portion 30 is greater than the thickness of the second side panel 13. The relatively thin first side panel 12 and second side panel 13 improve breathability at the sides, preventing excessive stuffiness during wear and maintaining dryness and comfort. The thicker first abdominal compression portion 20 and second abdominal compression portion 30 provide stronger support to help shape the abdominal curve, thereby enhancing wearing comfort. Appropriate thickness design can effectively relieve pressure, avoiding excessive compression on the wearer's abdomen and back, making the wearing process more comfortable. The thickest back panel 11 enhances support and comfort, providing soft and comfortable backing.

In one embodiment, the elasticity of the first abdominal compression portion 20 is greater than that of the second abdominal compression portion 30. When worn, the second abdominal compression portion 30 is positioned on the inner layer, and the first abdominal compression portion 20 is positioned on the outer layer. The abdominal area is first flattened by the less elastic second abdominal compression portion 30 and then tightened by the more elastic first abdominal compression portion 20.

In one embodiment, at least one pull handle 31 is provided on the second abdominal compression portion 30. The function of the pull handle 31 is to facilitate the user in applying force during wear.

In one embodiment, two pull handles 31 are provided on the second abdominal compression portion 30, and the two pull handles 31 are located at different positions in the height direction of the second abdominal compression portion 30. The two pull handles 31 correspond respectively to convenient force application when the second abdominal compression portion 30 is detachably connected with the first compression band 21 and the second compression band 22.

In one embodiment, the second abdominal compression portion 30 substantially covers the entire front abdominal region of the user. This design is convenient, allowing the user to first use the second abdominal compression portion 30 to flatten the abdomen.

In one embodiment, the hook-and-loop fastener 51 on the second abdominal compression portion 30 substantially covers the entire front abdominal region of the user. With this design, the detachable connection area between the first abdominal compression portion 20 and the second abdominal compression portion 30 is larger, providing a wider adjustment range suitable for different postpartum stages.

In one embodiment, the first abdominal compression portion 20 is provided with a hook surface 52 of the hook-and-loop fastener, where the height of the hooks on the hook surface 52 is less than the thickness of the first abdominal compression portion 20. By setting the hook height smaller, friction with the user is reduced, thereby increasing comfort.

In one embodiment, the edge of the second abdominal compression portion 30 maintains a certain distance from the edge of the hook-and-loop fastener 51 on the second abdominal compression portion 30. This arrangement prevents the edges of the abdominal binder from rolling backward, thereby improving the fit and comfort of the edges.

In one embodiment, the hook-and-loop fastener 51 on the second abdominal compression portion 30 is sewn onto the second abdominal compression portion 30 by stitching, wherein the stitching comprises a first stitch 511 located at the edge region of the hook-and-loop fastener 51 and a second stitch 512 located in the middle region of the hook-and-loop fastener 51. The second stitch 512 is wave-shaped. By setting the second stitch 512 in a wave shape, when the second abdominal compression portion 30 stretches laterally, the second stitch 512 will not break.

The present embodiment of the abdominal binder comprises: a waist support portion, arranged to be positioned at the waist region of the user when worn and extending toward both sides of the user; a first abdominal compression portion connected to a first side 18 of the waist support portion, configured to extend along the abdomen when worn; a second abdominal compression portion connected to a second side 19 of the waist support portion, configured to extend along the abdomen when worn; the first abdominal compression portion and the second abdominal compression portion are detachably connected and provide compression force to the user's abdomen when connected; and a back support member arranged on the waist support portion. The back support member comprises at least one support strip, which extends along the user's height direction when the abdominal binder is worn. The support strip gradually bends and extends from both ends 81, 82 toward the center 90 along the direction close to the user's back. The center 90 of the support strip can provide stronger support to increase comfort. Meanwhile, due to the support force applied to the back by the support strip, the reaction force enhances the compression force provided by the connection between the first abdominal compression portion and the second abdominal compression portion to the user's abdomen, thereby improving the abdominal shaping effect.

To make the objectives, technical solutions, and advantages of the present application clearer, the following further describes the present application in detail with reference to the accompanying drawings and embodiments. It should be understood that the specific embodiments described herein are merely intended to explain the present application and are not intended to limit the present application.

The above descriptions are only specific implementations of the present application. It should be noted that, for those of ordinary skill in the art, various modifications and refinements can be made without departing from the principles of the present application. These modifications and refinements should also be regarded as falling within the protection scope of the present application.

What is claimed is:

1. An abdominal binder, comprising:
a waist support portion configured to be positioned in a waist region of a user and to extend laterally to both sides of the user when worn, wherein the waist support portion is at least partially made of a sheet material, the sheet material comprising a first outer layer, a second outer layer, and a bonding layer disposed between the first and second outer layers, wherein the bonding layer comprises a first adhesive sub-layer, a cushioning layer, and a second adhesive sub-layer sequentially laminated from the inner side to the outer side;
a first abdominal compression portion connected to a first side of the waist support portion and configured to extend along an abdominal region of the user when worn;
a second abdominal compression portion connected to a second side of the waist support portion and configured to extend along the abdominal region of the user when worn, wherein the first abdominal compression portion and the second abdominal compression portion are detachably connected to each other and, when connected, provide a compressive force to the abdominal region of the user; and
a back support member disposed on the waist support portion, the back support member including at least one support strip extending in a height direction of the user when the abdominal binder is worn, the at least one support strip having a center and being gradually curved from both ends toward the center in a direction approaching a user's back, wherein the back support member is embedded in the cushioning layer.

2. (canceled)

3. The abdominal binder of claim 1, wherein the least one support strip gradually decreases in thickness from the center toward both ends.

4. The abdominal binder of claim 1, wherein the back support member further comprises connecting strips connected between adjacent support strips.

5. The abdominal binder of claim 4, wherein the connecting strips comprise at least a first connecting strip and a second connecting strip, the first and second connecting strips being disposed at different positions along the height direction of the user when the abdominal binder is worn.

6. (canceled)

7. (canceled)

8. (canceled)

9. (canceled)

10. The abdominal binder of claim 1, wherein the cushioning layer is a sponge layer.

11. The abdominal binder of claim 1, wherein the second outer layer is provided with ventilation holes.

12. The abdominal binder of claim 1, wherein the first abdominal compression portion comprises at least a first compression band and a second compression band, the first and second compression bands being disposed side by side and configured to extend transversely along the abdominal region when worn, the first and second compression bands being configured to be independently detachably connected to the second abdominal compression portion, and each of the first and second compression bands being independently adjustable in tightness by changing its connection position with the second abdominal compression portion.

13. (canceled)

14. The abdominal binder of claim 1, wherein an upper edge of the at least one support strip is arc-shaped.

15. The abdominal binder of claim 1, wherein a lower edge of the at least one support strip is arc-shaped.

16. (canceled)

17. (canceled)

18. (canceled)

19. (canceled)

20. (canceled)

21. An abdominal binder, comprising:
a waist support portion configured to be positioned in a waist region of a user and to extend laterally to both sides of the user when worn, the waist support portion is at least partially made of a sheet material, the sheet material having a cushioning layer disposed internally therein;
a first abdominal compression portion connected to a first side of the waist support portion and configured to extend along an abdominal region of the user when worn;
a second abdominal compression portion connected to a second side of the waist support portion and configured to extend along the abdominal region of the user when worn, wherein the first abdominal compression portion and the second abdominal compression portion are detachably connected to each other and, when connected, provide a compressive force to the abdominal region of the user; and
a back support member disposed on the waist support portion, wherein the back support member is embedded in the cushioning layer.

22. The abdominal binder of claim 21, the back support member including at least one support strip extending in a height direction of the user when the abdominal binder is worn, the at least one support strip having a center and being gradually curved from both ends toward the center in a direction approaching a user's back.

23. The abdominal binder of claim 22, wherein the least one support strip gradually decreases in thickness from the center toward both ends.

24. The abdominal binder of claim 22, wherein the back support member further comprises connecting strips connected between adjacent support strips.

25. The abdominal binder of claim 22, wherein the connecting strips comprise at least a first connecting strip and a second connecting strip, the first and second connecting strips being disposed at different positions along the height direction of the user when the abdominal binder is worn.

26. The abdominal binder of claim 21, wherein the cushioning layer is a sponge layer.

27. The abdominal binder of claim 22, wherein an upper edge of the at least one support strip is arc-shaped.

28. The abdominal binder of claim 22, wherein a lower edge of the at least one support strip is arc-shaped.
